(19) United States
(12) Patent Application Publication
(10) Pub. No.: US 2024/0349997 A1
(43) Pub. Date: Oct. 24, 2024

(54) FIBERSCOPE FOR STEREOSCOPIC IMAGING AND METHOD FOR ACQUIRING STEREOSCOPIC IMAGE DATA

(71) Applicant: Carl Zeiss Meditec AG, Jena (DE)

(72) Inventors: Christoph Hauger, Aalen (DE); Christoph Schaeff, Aalen (DE); Fang You, Aalen (DE)

(21) Appl. No.: 18/640,940

(22) Filed: Apr. 19, 2024

(30) Foreign Application Priority Data
Apr. 19, 2023 (DE) 10 2023 109 944.2

(51) Int. Cl. A61B 1/07

(52) U.S. Cl. CPC A61B 1/07

(57) ABSTRACT

A fiberscope for stereoscopic imaging has at least one wavefront manipulator which, for creating a sample beam, is configured to pre-shape a wavefront of the light from a light source such that the pre-shaped light is focusable substantially on an object point in an object region and raster-deflectable to a multiplicity of object points. The fiberscope also includes an illumination fiber for supplying the pre-shaped sample beam to the object region, and a detector fiber for supplying scattered light reflected and/or scattered at the respective object point to a detector which captures the scattered light and is connected to a computer unit. The computer unit is configured to compose the stereoscopic image from the captured scattered light. A method is for acquiring stereoscopic image data from a fiberscope.

### CROSS-REFERENCE TO RELATED APPLICATIONS

This application claims priority of German patent application no. 10 2023 109 944.2, filed Apr. 19, 2023, the entire content of which is incorporated herein by reference.

### TECHNICAL FIELD

The disclosure relates to a fiberscope for stereoscopic imaging. The disclosure also relates to a method for acquiring stereoscopic image data.

### BACKGROUND

Microsurgical methods are finding increased use, especially for sensitive tissue, for example within the eye in the field of ophthalmic surgery, but also for neurosurgical interventions. In ophthalmic surgery in particular, surgical microscopes which are used frequently for procedures in the anterior eye segment but also for procedures on the retina are increasingly being replaced by endoscopes which ultimately facilitate imaging in minimally invasive procedures.

In this context, cataract surgery is a frequent procedure in the anterior eye section, while epiretinal membrane peeling represents a typical procedure on the retina. In this context, epiretinal membrane peeling becomes necessary if there is unchecked growth of connective tissue cells on the surface on the retina, similar to scarring of the skin, on account of different diseases of the eye fundus. Causes for this unchecked cell proliferation include injuries and previous operations, but also laser treatments on the eye, already cured inflammations or else perfusion disorders of the retina. The proliferating cells ultimately form a mechanically stable cell aggregation in the form of what is known as an epiretinal membrane. While the patient is hardly impaired during an early stage thereof, the membrane contracts in later stages. Since the membrane rests securely on the retina, this contraction leads to the retina becoming ever more distorted, whereby folds arise at the location of sharpest vision, with this being referred to as a “macular pucker”. By this stage, the patient notices a reduced visual acuity and objects fixated on are perceived in increasingly distorted fashion. To rectify this, the vitreous humor in the eye is first of all surgically removed by a vitrectomy. This is followed, likewise in a surgical procedure, by the removal of the membrane from the retina via epiretinal membrane peeling. The vision can frequently be improved considerably as a result, and the distortions frequently also largely regress.

The object of the therapy always is to remove as many membrane parts as possible in order to eliminate the epiretinal contractions. In this context, barrel tweezers or vitreous tweezers are usually used for the membrane mobilization. These do not have a uniform configuration but are available in numerous straight and angled embodiments with different configurations. However, so-called “scrapers” are also used as an additional aid within the scope of membrane mobilization; these allow the surface to be scraped open, especially if the membranes adhere very strongly, in order to then be able to grasp the membrane parts mobilized thus using the tweezers. Moreover, specific needles, frequently referred to as “picks”, are also used to pierce or rip open the membrane. The membrane mobilization opens the closed surface of the membrane, and this leads to a reduction in the surface tension so that protruding lips of the membrane can be grasped by the tweezers in order to ultimately obtain a point of attack for membrane peeling. However, epiretinal membranes that have already existed for a long time often have pronounced retinal adhesions and localized retinal atrophies, wherefore the reliable and complete removal while avoiding the risk of retinal defects requires particular care. Equally, direct surgical complications arise relatively frequently in the process. In this context, particular mention should be given to retinal defects, which might for example arise due to instrument movement, but also to retinal detachment and tears, which generally arise during the membrane mobilization.

From the example of the above-described epiretinal membrane peeling on the retina, the need for the provision of improved imaging has become clear, in particular imaging that allows the membrane and possible lips in the membrane to be imaged reliably in order to allow the surgeon to perform epiretinal membrane peeling as safely as possible. However, the need for a three-dimensional representation, in particular, has also been become apparent in this context, in order to ensure that the membrane is distinguishable from the underlying retina so that damage to the retina is avoided. However, the need for stereoscopic imaging which provides the surgeon with depth information is also apparent in further microsurgical applications, especially in neurosurgery.

Stereoscopic endoscopes are already known from the prior art. For example, U.S. Pat. No. 7,751,694 B2 has already disclosed a stereoscopic endoscope in which an image sensor records two-dimensional images of an object or a scene with a plurality of focal planes or focus planes, which are displaced by changing the focal length of a “micromirror array lens” (MMAL) with a variable focal length. In that case, the image processing unit essentially extracts the sharpest pixels or regions from each two-dimensional image in order to ultimately obtain therefrom an appropriately sharp image with depth information. Appropriate depth information can be extracted therefrom on the basis of the known focal length of the respective two-dimensional image. In other words, the focal length of the MMAL of the endoscope known from U.S. Pat. No. 7,751,694 B2 is modified in such a way that each part of the examination object is essentially imaged sharply at least once. By virtue of compiling this information, it is possible to derive three-dimensional information about the examination object therefrom.

However, it was found to be disadvantageous that this method is very complicated and the apparatus, on account of its structure, is not suitable for use in a minimally invasive microsurgical method. For example, in posterior segment surgery the diameters of the surgical endoscopes used have in particular been continuously reduced in recent years. Thus, at this point in time, surgical endoscopes with light guides that have a diameter of merely 25 gauge or 27 gauge are used; this would not be realizable using the endoscope known from U.S. Pat. No. 7,751,694 B2.

The prior art, more precisely the publication Leite, Ivo T., et al. “Observing distant objects with a multimode fiber-based holographic endoscope.” APL Photonics 6.3 (2021):036112 (DOI: 10.1063/5.0038367), has disclosed a holographic fiberscope which uses the principle of raster scanning. In that case, images are reconstructed from the local response of an examination object—that is, ultimately the scattered light—to a sample beam which was pre-shaped by a micromirror actuator and transmitted via an illumination fiber in the form of a multimode fiber. The micromirror actuator, often alternatively also referred to as a DMD (“digital micromirror device”), uses a mirror array based on microelectromechanical system component technology (“MEMS technology”). In this context, the mirrors are actuatable on an individual basis and tiltable in particular. The micromirror actuator is irradiated by a light source in this case and pre-shapes the wavefront reflected by the micromirror actuator before the pre-shaped light is subsequently input coupled into the illumination fiber in order—in conjunction with the multimode fiber—to be able to define the position of the focus within the object region. In this case, the micromirror actuator influences the phase angle of the wavefront and is therefore also referred to as a holographic modulator.

In this case, the holographic endoscope—as already explained above—uses the principle of raster imaging, in which images are reconstructed from the scattered and/or reflected scattered light of an object or examination object irradiated by the sample beam which was pre-shaped by the micromirror actuator. In this case, the detector fiber collects scattered light that was scattered and/or reflected by the surface of the examination object, with the amount of scattered light depending on the local reflectivity of the object, the roughness, the alignment and the axial depth. The corresponding imaging region is spaced apart from the surface of the distal end of the illumination fiber and detector fiber. In the case of raster imaging, the light is focused on an object point on the surface of the examination object by way of the micromirror actuator, wherein the position of the focal plane, that is, ultimately the working distance, can be set by a suitable actuation of the micromirror actuator. This ensures that the imaging region, that is, the focusable region, is located in a plane with the object region. Ultimately, the light propagation through the illumination fiber is characterized empirically by way of a transmission matrix which describes the linear relationship between advantageously chosen sets of input and output fields. For example, it is possible to use the representation of orthogonal, plane waves cut off by the micromirror actuator as a basis for the input fields and diffraction-limited focal points in a square grid in the far-field plane of the distal fiber facet as a basis for the output fields. Following detection, the transfer matrix contains the information for configuring the binary micromirror actuator patterns for pre-shaping the proximal wavefront that leads to far field foci at the distal end of the endoscope. This allows tailored imaging regions to be scanned without having to move the multimode illumination fiber. Then, the registered intensities can be merged to form a common image. However, it was found to be disadvantageous that it is not possible to make stereoscopic recordings using the fiberscope described in the article by Leite, Ivo T., et al.

### SUMMARY

Accordingly, it is an object of the present disclosure to reduce the aforementioned disadvantages and in particular of providing a precise endoscope which is as compact as possible and via which it is possible to capture stereoscopic recordings of an examination object, and of providing a corresponding method for capturing stereoscopic pictures.

The aforementioned object is achieved by various embodiments of the disclosure.

A first aspect of the disclosure relates to a fiberscope for stereoscopic imaging. The fiberscope according to the disclosure includes at least one wavefront manipulator which, for creating a sample beam, is configured to pre-shape a wavefront of the light from a light source such that the pre-shaped light is focusable substantially on an object point in an object region and raster-deflectable to a multiplicity of object points. Moreover, the fiberscope includes at least one illumination fiber for supplying the pre-shaped sample beam to the object region and at least one detector fiber for supplying scattered light reflected and/or scattered at the respective object point to a detector which captures the scattered light. The detector is connected to a computer unit. The wavefront manipulator is also configured to create temporally and/or spectrally separated sample beams, which make a fixed stereo angle with each other.

Alternatively, the wavefront manipulator, the illumination fiber and the detector fiber are each provided twice. In this case, the wavefront manipulators then are further configured to create temporally and/or spectrally separated sample beams. In this case, the computer unit is always configured to compose the stereoscopic image from the captured temporally and/or spectrally separated scattered light.

A further aspect of the disclosure likewise relates to a fiberscope for stereoscopic imaging. In this case, the fiberscope includes a wavefront manipulator which, for creating a sample beam, is configured to pre-shape a wavefront of the light from a light source such that the pre-shaped light is focusable substantially on an object point in an object region and raster-deflectable to a multiplicity of object points. Moreover, the fiberscope includes an illumination fiber for supplying the pre-shaped sample beam to the object region. Moreover, the fiberscope includes at least two detector fibers for supplying scattered light reflected and/or scattered at the respective object point to a respective detector which captures the scattered light. The detector is connected to a computer unit configured to compose the stereoscopic image from the captured scattered light.

Additionally, a further aspect of the disclosure relates to a method for acquiring stereoscopic image data from a fiberscope. The method includes the following steps:
a) pre-shaping a wavefront of the light from a light source via at least one wavefront manipulator such that, for the purpose of creating a sample beam, the pre-shaped light can be focused substantially on an object point in an object region and can be raster-deflected to a multiplicity of object points,
b) supplying the pre-shaped sample beam to an object region via at least one illumination fiber,
c) focusing the supplied light on an object point in the object region by way of the wavefront manipulator,
d) supplying the scattered light reflected or scattered at the object point to a detector via a detector fiber,
e) repeating steps c) and d) for at least some of the object points in the object region, and
f) extracting a stereoscopic image from the acquired data of the detector.

In general, the term “light source” relates to an apparatus embodied to emit light at a specific wavelength or in a specific wavelength range. However, within the scope of the present disclosure, the term “light source” is also understood to mean an apparatus that is able to emit light at a plurality of wavelengths and/or in a plurality of wavelength ranges. In this context, the term “light” includes not only visible light but also infrared light and ultraviolet radiation.

In general, the term “wavefront manipulator” relates to an apparatus used to shape the wavefront of the light emitted by the light source before this light is input coupled into the illumination fiber. In a particularly preferred embodiment, the wavefront manipulator is in the form of a micromirror actuator, that is, includes an arrangement of a multiplicity of individual mirrors which are actuatable on an individual basis in order to appropriately pre-shape the light incident thereon; this is described in detail in the publication by Leite, Ivo T., et al. set forth at the outset. In other words, the micromirror actuator—often also referred to as “digital micromirror device”(abbreviated DMD)—can in a targeted manner pre-shape the wavefront of the light which comes from the light source and is reflected by the micromirror actuator, in order to modify the properties of the light reflected by the micromirror actuator. In combination with the illumination fiber, this ultimately allows a targeted adjustment of the properties of the light which, coming from the illumination fiber, is incident as sample beam on the examination object. In particular, it is possible to set both the position of the focal plane and the position of the focal point within the focal plane in the process. Thus, this allows the position of the focus to be modified-without having to move the illumination fiber to this end. Since such wave manipulators can be actuated very quickly, the use of the wave manipulator allows the object region to be raster-illuminated, that is, the focus can be allowed to migrate over the examination object in the manner described in the publication by Leite, Ivo T., et al. set forth at the outset. In this case, a controller is provided for the actuation of the wavefront manipulator, with the term “controller” generally relating to an apparatus which can be used for the targeted actuation of in particular the wavefront manipulator or the individual mirror elements of the micromirror actuator. Moreover, the controller may however also be used to control other elements, for example the detector, that is, influence the functionality thereof directly or indirectly. To pre-shape the wavefront in this context, a controller can be dedicatedly assigned to each wavefront manipulator, or the wavefront manipulators can be actuated by a common controller.

In general, the term “illumination fiber” denotes the fiber serving to supply the light which was pre-shaped by the wavefront manipulator to the examination object. This light which was pre-shaped by the wavefront manipulator and ultimately is incident in focus on the surface of the examination object is generally also referred to as a “sample beam”. In this context, the illumination fiber has a first, proximal end and a second, distal end. In the present case, the term “proximal end” relates to the end of the illumination fiber which is close to the wavefront manipulator and serves to input couple the light which was pre-shaped by the wavefront manipulator. By contrast, the term “distal end” in the present case relates to the end of the illumination fiber which is close to the examination object and from which the sample beam re-emerges from the illumination fiber and is guided to the examination object. Instead of the term “fiber”, the term “light guide” can also be used synonymously.

In general, the term “object region” is understood to mean the portion of the surface of the examination object which can be irradiated or illuminated by the sample beam from the fiberscope. The size of this object region depends firstly on the numerical aperture but in particular also on the “working distance”, that is, ultimately on the distance of the distal end of the illumination fiber from the object region or surface of the examination object. In this context, attention is also drawn to the fact that the term “object point” relates to a single point within the object region on which the sample beam which was pre-shaped by the wavefront modulator can be focused. In this case, the number of illuminated object points is ultimately a measure for the resolution with which the stereoscopic images are captured. For example, the working distance can be measured via optical coherence tomography (abbreviated “OCT”). A coherent light beam typically in the near infrared range is created in this context and can be guided to the object region through the illumination fiber. The time of flight of the light can be recorded by measuring the phase shift vis-à-vis a reference beam, and hence the OCT measurements render it possible to measure distances, which are expressed in different phase shifts of the light received from the surface of the object region vis-à-vis the reference beam. In particular, the illumination fiber or an additional fiber can be used for the OCT measurement in this case. In an alternative or in addition, it is also possible in particular to use the sample beam provided for the illumination provided that it has the properties required for the OCT measurement and provided that it is split into a sample beam and a reference beam prior to incidence on the wavefront manipulator such that the reference beam can be superimposed on a light which is returning from the object region and connected to the sample beam. The reference beam can thus be split from the sample beam proximally in front of a wavefront manipulator. In particular, the OCT imaging modality can be used in ophthalmology for the purpose of examining the retina and the anterior eye segment, and also in cardiology and dermatology.

Within the scope of the present disclosure, the term “detector fiber” relates to the light guide which can be used to capture the light reflected and/or scattered by the respective object point—also referred to as “scattered light”—and guide the latter to the detector. Here, the detector fiber likewise has a first, proximal end and a second, distal end. In this case, the “proximal end” of the detector fiber is the end serving to output couple the scattered light to the detector. By contrast, the “distal end” of the detector fiber denotes the end serving to input couple the scattered light into the detector fiber. In this case, the detector fiber has a capturing region, the size or diameter of which is determined by the so-called acceptance angle, which is specific to each light guide, and the observation distance. In this case, the “observation distance” ultimately is the distance between the distal end of the detector fiber and the examination object. In this case, the observation distance is preferably substantially identical to the working distance. If scattered light is incident on the distal end of the detector fiber outside of the acceptance angle, then this scattered light is not guided to the detector. In this context, attention is also drawn to the fact that the capturing region need not necessarily be identical to the object region but usually has a large overlap region therewith. In this context, the term “detector” is understood to mean a photodetector, a light sensor or an optical detector in particular, that is, electronic components which convert scattered light into an electrical signal or which indicate an electrical resistance that is dependent on the incident radiation. Within the scope of the disclosure, the detector captures the scattered light which is reflected and/or scattered by the surface of the examination object and supplied to the detector through the detector fiber.

The signals acquired by the detector or detectors are merged to form a stereoscopic image by a computer unit. Within the scope of the disclosure, the phrase “computer unit” in this context includes in particular a computer suitable for merging the individual signals from the detector or detectors to form the stereoscopic image. In this case, the computer unit may also be part of the control unit or else include the latter. Provision is also made for at least parts of the computer unit to be situated at different locations or for the stereoscopic images to be composed from the detector data via the Internet and in equipment-independent fashion within the scope of “cloud computing”.

Thus, according to the disclosure, stereoscopic images can be created in different ways. In this case, according to the disclosure, a preferred embodiment envisages that the wavefront manipulator, the illumination fiber and the detector fiber are each provided twice. In this case, the wavefront manipulators are configured to create temporally and/or spectrally separated sample beams. Moreover, the computer unit is also configured to compose the stereoscopic image from the temporally and/or spectrally separated scattered light. In this case, the two illumination fibers each illuminate object points in different, albeit overlapping object regions on the surface of the examination object. In this case, the two sample beams are temporally and/or spectrally separated. The scattered light from the individual object points in each case input coupled into the distal ends of the detector fibers is in each case guided by the detector fibers to a corresponding detector and captured there. By virtue of the two wavefront manipulators being actuated such that the individual object points in the object regions are in each case raster-scanned by a focused sample beam and the scattered light for each object point is captured by the corresponding detector, it is ultimately possible to create two frames—left frame and right frame—from the signals acquired by the two detectors, and these two frames can be composed into a stereoscopic image of the overlapping object regions by the computer unit. To prevent an impairment of the signal acquisition, it has proven its worth in this setup for the individual object points of the object regions to be illuminated sequentially, that is, in temporally separated manner, by the two illumination fibers. In general, the term “temporal separation” relates to only one of the two sample beams irradiating the object region at any one time, with the other sample beam being deactivated at this time. In this context, the sample beams are preferably switched on and off in alternation. In this case, the detectors are embodied such that these only capture the scattered light from the corresponding sample beam in each case, while the scattered light from the interfering other sample beam is not taken into account when creating the respective frame. However, it is alternatively also possible to carry out a “spectral separation” here, that is, use light sources at different wavelengths. In this case, appropriate filter elements are assigned to the detector fibers and/or the detectors themselves in order to prevent scattered light at the “wrong” wavelength from being supplied to the corresponding detector. However, within the scope of the disclosure provision can also be made for the detector to be configured to identify the interfering signal and disregard this during the signal acquisition. Moreover, it could be possible to make do without the second illumination fiber in the case of the spectral separation, and the spectrally separated sample beams could be guided to the object region using only one illumination fiber.

The light source can also be provided twice if two wavefront manipulators are used, with each of the two light sources illuminating a respective corresponding wavefront manipulator in this case. In this case, the two light sources can emit light with the same spectral distribution or with different spectral distributions. However, the scope of the disclosure also provides for the use of only one light source, which is used for both wavefront manipulators by way of a beam splitter, preferably a semi-transparent beam splitter.

In an alternative embodiment, the fiberscope according to the disclosure includes only one wavefront manipulator, one detector fiber and also only one illumination fiber. In this case, the wavefront manipulator is configured to create temporally and/or spectrally separated sample beams, which make a fixed stereo angle with each other. The computer unit is also configured to compose the stereoscopic image from the captured temporally or spectrally separated scattered light. In this case, the wavefront manipulator is actuated such that all raster-scanned object points in the object region are always raster-scanned by two sample beams which make a defined stereo angle with each other. However, this case requires a temporal separation of the two frames, that is, ultimately of the left image from the right image, in order to create the stereo impression. Consequently, both the fiberscope according to the disclosure and the method according to the disclosure can be realized in a particularly simple solution with only one illumination fiber and also only one detector fiber. However, a spectral separation can also be implemented in an alternative or in addition, wherein, however, the availability of a second detector fiber was found to be advantageous in this case. In this case, both detector fibers or the corresponding detectors themselves contain appropriate filter elements, which are also used in the above-described embodiment that makes use of two light sources with different spectral properties. However, the stereoscopic image can alternatively also be captured with the aid of structured light. To this end, the object region is illuminated using a defined light pattern. In this case, this light pattern may consist of a plurality of strips, optionally of different widths, or of a multiplicity of points which have defined distances from one another. In this case, the strips can be created via a targeted positioning of the sample beam on the object surface. In this case, preference is given to the provision of a plurality of strips which are separated from one another by non-illuminated strips such that, ultimately, a light pattern with alternating light and dark strips is created. If the light pattern is formed by individual light spots, then it was found to be particularly preferable for the light spots to have an equidistant spacing from one another. The shape and/or the position of the light pattern is preferably modifiable. If this structured light is incident on a surface in non-perpendicular fashion, for example in the case of an irregularly shaped surface, then there is a deformation of the light pattern which can then be detected by the detector. However, this then requires the detector fiber and the detector corresponding thereto to be configured to register the origin of the scattered and/or reflected scattered light in addition to its intensity, that is, register the coordinates of this scattered light in order to herewith establish a correlation with the position of the object points. It is possible to make do without the aforementioned correlation if the light pattern is formed as a point pattern. As a result of this spatially resolved detection, it is possible to register the disturbance in the light pattern induced by the surface and thus possible to deduce the depth structure, that is, the depth profile in particular, of the examination object. Then, these data can be used—in a manner known to a person skilled in the art—to create stereoscopic images, for example via triangulation in the case of a known offset between the distal ends of illumination fiber and detector fiber. In this context, the term “offset” is generally understood to mean the relative position and orientation in particular. Alternatively, a “time of flight” measurement can also be used to measure the time of flight required by scattered light scattered and/or reflected at an object point to be guided to the detector. From time-of-flight differences measured in the process, it is then also possible to extract information regarding the surface of the examination object, and this can then be used to again obtain depth information. As described above, this depth information can then be used in turn to create stereoscopic images. Here, too, the sample beam can then be directed at the individual object points in a targeted manner by the targeted pre-shaping of the wavefront. The depth information obtained herewith represents a 3-D representation of the surface of the object region of the examination object. This 3-D representation can be used for example to perform robot-assisted procedures, for example to define forbidden regions within the sense of a “no-fly zone”.

In a further alternative embodiment of the disclosure, the wavefront manipulator and the illumination fiber are each provided once and the detector fiber is provided at least twice. In this case, the detector fibers have a fixed, lateral spacing from one another and are each configured to supply the scattered light to a corresponding detector. The computer unit is further configured to compose the stereoscopic image from the captured scattered light. In this embodiment, the scattered light scattered and/or reflected by the respective object point is captured simultaneously by the two detector fibers and supplied to the corresponding detector. The signals of the scattered light acquired separately for the two detector fibers can then be combined by calculation to form a stereoscopic image by the computer unit. In this context, it is also proven its worth if, ultimately, there is also a provision of two detectors which are coupled to one of the detector fibers in each case. As a result, it is particularly easy to simultaneously capture the scattered and/or reflected scattered light input coupled into the detector fibers; ultimately, this has a positive effect on the duration required to capture the stereoscopic images. The lateral spacing of the two detector fibers forms the stereo basis and hence also determines the quality of the stereo impression. As yet to be explained below, this is because the quality of the stereo impression depends significantly on the ratio of the stereo basis, that is, the lateral spacing of the distal ends of the detector fibers, and the observation distance, that is, the distance of the capturing region on the examination object from the distal ends of the detector fibers.

As a result, a fiberscope can be provided easily, the latter allowing a surgeon to create stereoscopic recordings of an operating site, for example in order to obtain the desired results in the case of an ophthalmological procedure. Moreover, this also makes it possible to use very small fiber diameters. Furthermore, this provides a simple method allowing the capture of stereoscopic images.

It was also found to be advantageous if the detector fibers have a fixed lateral spacing from one another. In this context, it has then further proven its worth for the (lateral) spacing between the two detector fibers to lie between 50 μm and 500 μm, preferably between 100 μm and 400 μm and particularly preferably between 150 μm and 300 μm and particularly preferably be 200 μm. In this case, the phrase “spacing between the two detector fibers” is generally understood to mean the lateral spacing between the two distal ends of the detector fibers. This spacing ultimately forms the stereo basis. In this context, it is determined perpendicular to the—parallel—longitudinal axes of the detector fibers. As already mentioned above, the suitable choice of the stereo basis influences the quality of the stereoscopic impression of the fiberscope. Thus, the stereoscopic impression in the case of for example a surgical microscope is determined by the ratio of stereo basis to focal length of the main objective. In this context, a ratio of 1:10 was found to be sensible in order to create a good stereoscopic impression. Since usual working distances or focal lengths of the fiberscope are between 0.5 mm and 3 mm in the present case, a good stereo impression can be obtained with the aforementioned values of the stereo basis. Since the lateral spacing of the detector fibers and hence however also the stereo basis are unchangeable, the stereoscopic impression suffers if there is too great a change in the working distance. The availability of additional detector fibers has then particularly proven its worth in this case. In this case, these additional detector fibers are arranged in such a way that their distal ends have a larger or smaller stereo basis than that of the other detector fibers. In other words, the detector fibers can now be combined in pairs which have different stereo bases. If there now is a change in the observation distance/working distance, then it is possible to use the suitable detector fibers for capturing the scattered light scattered or reflected by the object region. For example, detector fibers with a smaller stereo basis can be used if there is a reduction in the working distance. By contrast, detector fibers whose distal ends have a greater lateral spacing from one another can be used if there is an increase in the working distance. In particular, the pairing of detector fibers can also be selected dynamically, with the result that a fine gradation of possible stereo bases is available, especially in the case of a plurality of detector fibers, and hence a good stereo impression can be obtained over a large working distance range.

As already mentioned above, it was found to be advantageous if the detector is provided twice, especially when using a plurality of detector fibers. As a result, a dedicatedly assigned detector can be provided for each detector fiber, the detector capturing the scattered and/or reflected scattered light supplied by the corresponding detector fiber. However, it is alternatively also possible to use only one detector in this context. However, in that case the scattered light from the detector fibers must be captured in temporally separated fashion, that is, ultimately in alternation and with a time offset, in order to achieve a clean separation of the signals from the two detector fibers.

Moreover, if two detector fibers are used, it was found to be particularly advantageous within the scope of the disclosure if the two detector fibers are arranged such that substantially the same detection region within the object region is observable. Since ultimately the creation of overlapping frames is required for the creation of the stereoscopic images, it is a requirement that the capturing regions overlap at least in part. If the capturing regions are substantially the same, then this has a particularly positive influence on the stereoscopic observation and moreover also leads to the observable region on the examination object being able to be chosen to be as large as possible.

It was also found to be advantageous if the size of the object region that is raster-scannable is variable. Especially if the capturing region of the detector fiber(s) is greater than the object region, this ultimately makes it possible to be able to modify the raster-scanned region and hence also the size of the image capturable by the detector. Ultimately, this functionality then makes it possible to realize a stereo zoom function. For example, the latter can be used in a first step to raster scan a large object region of the examination object at only a low resolution, in which the object points thus have comparatively large distances from one another, in order to initially obtain an overview of the examination object. In a subsequent step, it is then possible to raster scan and capture a portion of the previously captured object region at a higher resolution by virtue of the spacing of the individual object points being reduced in this case. However, it is self-evident in this context that the need for the two detector fibers to have the same capturing region or at least an overlapping capturing region, from which the stereoscopic image is created, is unchanged.

What has also proven its worth in this context is for the wavefront manipulator to be also configured to vary the size of the object region when the working distance is changed. This enables an automatic adjustment of the object region such that this ensures that it is always the same object region that is illuminated, even in the case of a changing working distance. Moreover, this can ensure that the overlap region between the two object regions is always maintained. As already mentioned above, this working distance can be determined via OCT, for example. The distance information obtained thus can be used to implement the adjustment of the object region in a manner dependent on the working distance. In other words, the working distance between the distal end of the illumination fiber and the object region is used to adjust the size of the object region. The distance information can also be used to provide an autofocus functionality for the stereoscopic imaging. This ensures that the sample beam is always focused on the respective object point.

It was also found to be advantageous if the illumination fibers and/or the detector fibers are arranged immediately adjacent to one another. In general, the phrase “immediately adjacent” refers to the circumstances that the illumination fibers and/or the detector fibers are in direct contact with one another, at least in the region of their distal ends. In this case, the distal ends essentially open into one plane. If a plurality of illumination fibers and/or detector fibers are used, then it is possible for all fibers to be arranged immediately adjacent to one another. However, in a broader interpretation, this also includes configurations in which the illumination fibers and/or collector fibers have a spacing, albeit a small spacing, from one another or are spaced apart from one another by a separation element. In addition to a defined positioning, the immediately adjacent arrangement of the fibers offers the advantage here that the fiberscope according to the disclosure can be integrated more easily in a medical instrument and/or implant.

In this context, it has also proven its worth if a plurality of detection fibers and/or illumination fibers can be paired dynamically, that is, depending on the time or circumstances. In this context, paired in particular describes the state where the signals recorded by the paired detection fibers or output by the illumination fibers are related to one another, especially within the scope of the evaluation. For example, the signals of paired detection fibers can thus be used to create images which generate a stereoscopic impression depending on the spacing of the paired detection fibers.

It has also proven its worth for a plurality of detection fiber pairs and/or illumination fiber pairs to be used together so that the individual fibers of the respective paired fiber pairs have the same spacing from one another. In an alternative or in addition, it may be advantageous for the individual fiber pairs to have the same spacings from one another. As a result, the object region can be examined simultaneously over a large area, wherein a uniform impression of the examination object, especially a stereoscopic impression, can be obtained.

It was also found to be advantageous if the light source is embodied as a laser, particularly preferably as an RGB laser. A color reproduction of the stereoscopic images can be obtained by using an RGB laser in particular.

It was also found to be particularly advantageous if the at least one illumination fiber is formed as a multimode light guide. In particular, the transfer of the wavefront which was pre-shaped by the micromirror actuator to the examination object can be realized particularly simply by the use of multimode light guides. Thus, the light transport through optical multimode light guides has properties which differ from those of other complex media. Multimode light guides support a number of propagation invariant modes (PIMs), which do not change their field distribution during the propagation through the fiber and each of which is characterized by a specific propagation constant which determines its phase speed. However, since multimode light guides are comparatively expensive, forming the detector fiber(s) as single-mode light guides has also proven its worth in this context.

It has also proven its worth for a display apparatus for displaying the stereoscopic image to be provided. In particular, a 3-D monitor can be used in this context. The scope of the disclosure also provides for the endoscopic stereoscopic image to be used for augmenting another stereoscopic image, for example one created via a surgical microscope.

Within the scope of the method according to the disclosure, it was also found to be advantageous if the focusing in step c) is preceded by the determination of the working distance between the distal end of the illumination fiber and the object region. As already described above, this working distance may be determined via OCT, for example.

In an alternative or in addition, the method according to the disclosure may include selecting a combination of detection fibers or detection fiber pairs and/or illumination fibers after the determination of the working distance and preferably before the focusing.

Moreover, it was found to be particularly advantageous if an operating element is provided for actuating a controller which controls the wavefront manipulator. This allows the user, for example a surgeon, to start the image capture in targeted fashion. In this case, the operating element can be mounted in direct proximity to the wavefront manipulator. In an alternative or in addition, however, the operating element can also be arranged on a central operating unit. In this case, the operating element can be embodied as a pushbutton and/or as a digital solution, for example as an element on a touchscreen. Moreover, the operating element can be realized as a voice control facility.

It was found to be particularly advantageous for the application if the fiberscope according to the disclosure is at least partly integrated in a surgical instrument, preferably in an ophthalmic surgical instrument. In particular, in this case, the at least one wavefront manipulator and/or the controller thereof can be integrated in the surgical instrument. Moreover, in the context of the disclosure, provision is also made for the at least one light source likewise to be arranged in the surgical instrument. In an alternative, however, the light source can be coupled to the wavefront manipulator via a light guide such that the light of the light source is steered to the wavefront manipulator. Moreover, the operating element can also be arranged on the surgical instrument, thereby enabling the surgeon to directly operate the controller and thus ultimately also the wavefront manipulator in order for example to be able to manually start the capture of the stereoscopic images.

In an alternative or in addition, stereoscopic parameters of a user can also be used to influence the ratio between stereo basis and working distance. For example, it may be advantageous to increase or reduce the size of the stereo basis as a result of specifications, inputs or requirements of a user. As a result, the stereo basis can be adjusted flexibly to the situational or user-specific requirements. In this context, stereoscopic parameters of a user may also include the eye spacing of the user.

Furthermore, stereoscopic parameters of a user may also include values for the ratio of stereo basis to working distance used in the past, especially regularly recurring deviations from the ratio of 1:10, which was found to be sensible or advantageous, and averaged values or values calculated differently.

### BRIEF DESCRIPTION OF DRAWINGS

The invention will now be described with reference to the drawings wherein:

FIG. 1 shows a schematic view of a first embodiment of a fiberscope according to the disclosure;

FIG. 2 shows a detail view of the first embodiment of the fiberscope;

FIG. 3 shows a schematic illustration of the overlapping object regions of the fiberscope;

FIG. 4 shows a schematic view of a second embodiment of the fiberscope according to the disclosure;

FIG. 5 shows a detail view of the second embodiment of the fiberscope with a small working distance;

FIG. 6 shows a detail view of the second embodiment of the fiberscope with a larger working distance;

FIG. 7 shows a schematic view of a third embodiment of the fiberscope according to the disclosure;

FIG. 8 shows a detail view of the second embodiment of the fiberscope with a middling working distance;

FIG. 9 shows a detail view of the distal end of a fourth embodiment of the fiberscope in cross section; and,

FIG. 10 shows a flowchart of a method for capturing stereoscopic images.

### DETAILED DESCRIPTION

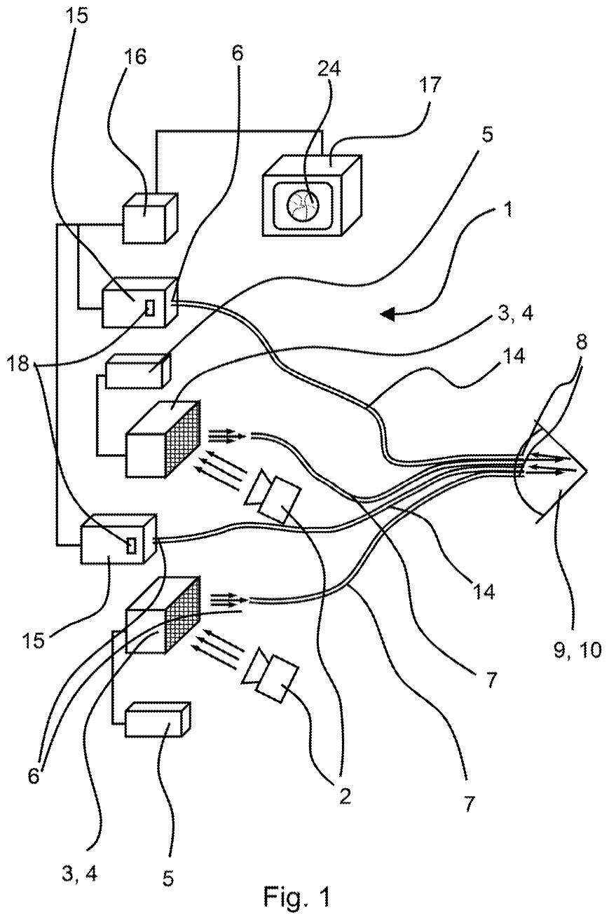

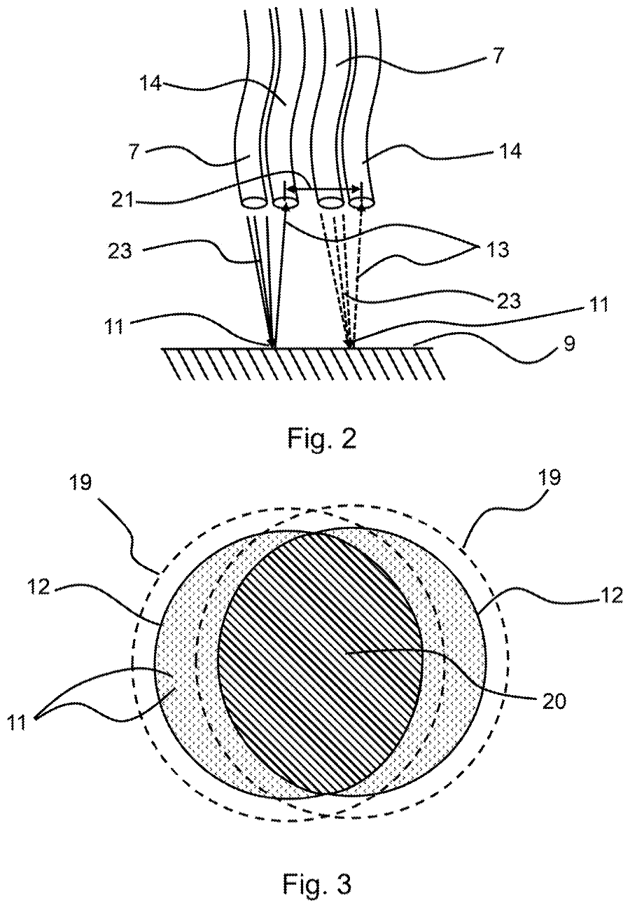

FIG. 1 shows a schematic view of a first embodiment of a fiberscope 1 according to the disclosure, which is configured for stereoscopic imaging. In the embodiment shown, the fiber endoscope 1 includes two light sources 2, each embodied as a laser light source 2 with different wavelengths. The two light sources 2 each emit light at a wavefront manipulator 3, which is formed as a micromirror actuator 4 in the embodiment shown. In this case, the micromirror actuator 4 is actuatable by a controller 5 for the purpose of pre-shaping the wavefront reflected by the micromirror actuator 4. In the embodiment shown, the light which was pre-shaped by the micromirror actuator 4 is in each case coupled into a proximal end 6 of an illumination fiber 7, the latter embodied as a multimode fiber whose distal ends 8 are directed at an examination object 9, an eye 10 of a patient in the embodiment shown. Suitable pre-shaping of the wavefront makes it possible to focus the light emitted by the illumination fiber 7 as a sample beam 23 on an object point 11 within an object region 12 formed on the examination object 9 and modify the position of the focus within this object region 12 in a targeted manner in order to ultimately illuminate the individual object points 11 on the object region 12 by the focused sample beam 23 and ultimately use the latter to scan the object region. As will still be explained in detail below with reference to FIG. 3, there is significant overlap of the object regions 12 of the two illumination fibers 7 in order to ensure that the scanned frames represent the same regions at least in part. The scattered light 13 reflected and/or scattered at each object point 11 in the respective object region 12 is input coupled into a distal end 8 of a detector fiber 14 and output coupled again at a proximal end 6 of the detector fiber 14, from where it is supplied to a detector 15. The detector 15 acquires the signals of the scattered light 13 reflected and/or scattered at the respective object point 11. In the embodiment shown, a total of two detector fibers 14 and two detectors 15 are provided. The acquired signals are subsequently composed to form a stereoscopic image 24 by a computer unit 16 and are displayed on a display device 17, a 3-D monitor in the present case. As will still be explained in detail below with reference to the detail view depicted in FIG. 2, the two light sources 2 emit spectrally separated light and ultimately have different wavelengths. Moreover, filter elements 18 are provided and configured such that only the reflected and/or scattered light 13 which is reflected and/or scattered by the object points 11 illuminated by the corresponding illumination fiber 7 at the appropriate wavelength is supplied to the corresponding detector 15. However, a time-offset raster scan can also be implemented in an alternative, with the result that the respective detector 15 only acquires the signals actually originating from the corresponding illumination fiber 7. In other words, each sample beam 23 ultimately has a dedicatedly assigned detector 15.

FIG. 2 shows a detail view of the distal ends 8 of the illumination fibers 7 and detector fibers 14 of the fiberscope 1 according to the first embodiment depicted in FIG. 1. As already explained above, the light sources 2 emit light at different wavelengths. Thus, as indicated by the dashed lines in FIG. 2, the light output coupled as sample beam 23 from the distal ends 8 of the illumination fibers 7 also has different wavelengths. The filter elements 18 assigned to the detector fibers 14 or the detectors 15 themselves in this case also ensure that only the scattered light 13 originating from the corresponding light source 2 is captured by the respective detector 15.

FIG. 3 shows, by way of example and also only by way of a simplified representation, the overlap of the two object regions 12, that is, the regions of the examination object 9 whose object points 11 can be raster-scanned by the respective sample beams 23 as a result of the suitable pre-shaping of the respective wavefront by the wavefront manipulator 3. Moreover, FIG. 3 also depicts the capturing regions 19 of the two detector fibers 14, that is, ultimately the regions from where reflected and/or scattered light 13 can be input coupled into the detector fiber 14 and supplied to the corresponding detector 15. In the embodiment shown, these capturing regions 19 are arranged concentrically with the object regions 12. In this case, the respective capturing region 19 of the detector fiber 14 is larger than the object region 12 of the illumination fiber 7. The overlap region 20 of the two illumination fibers 7 depicted by hatching in this case represents the region which can ultimately be depicted stereoscopically. The overlap of the two capturing regions 19 of the detector fibers 14 elucidates the necessity of temporally and/or spectrally separating the sample beams 23 from one another. Without this separation, the detectors 15 would each capture scattered light 13 from both sample beams 23.

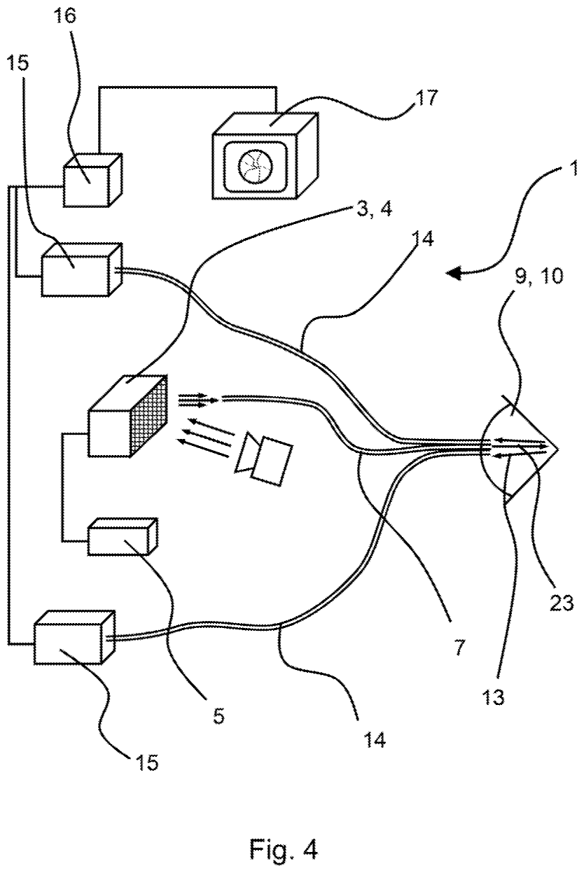

FIG. 4 shows a particularly preferred embodiment of the fiberscope 1. In this case, only one wavefront manipulator 3 is provided; it is irradiated by a light source 2 and the light pre-shaped thereby is then ultimately input coupled into the proximal end 6 of an illumination fiber 7. The light is supplied to the examination object 9, the eye 10 of a patient in the present case, through the illumination fiber 7 and emerges from the distal end 8 of the illumination fiber 7 as sample beam 23. The sample beam is focused on an object point 11 within the object region 12 as a result of the pre-shaping of the wavefront. The scattered light 13 which was scattered and/or reflected by this object point 11 is input coupled into the two detector fibers 14 which are arranged immediately adjacent to the illumination fiber 7 and have longitudinal axes that are spaced apart from one another at a fixed lateral spacing 21.

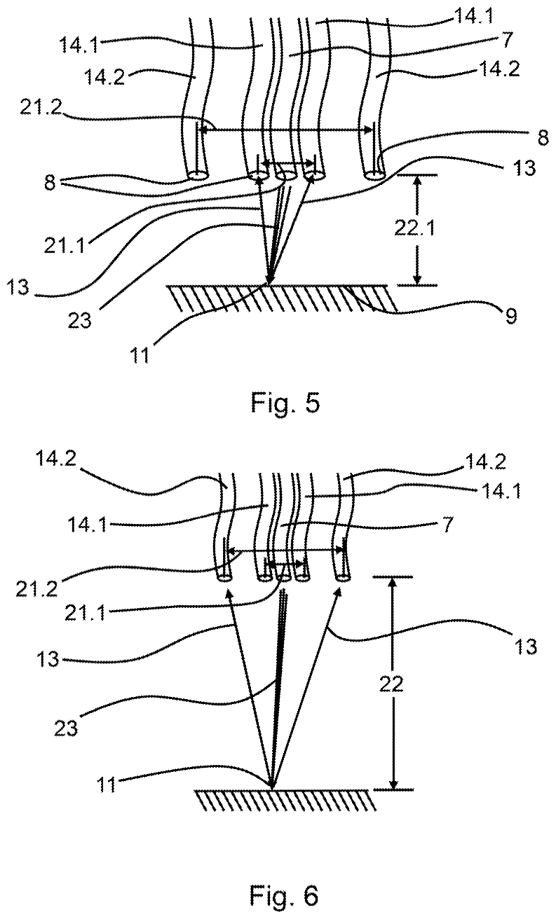

As may be gathered from the detail view depicted in FIG. 5 and FIG. 6 only, this embodiment includes not only the two first detector fibers 14.1, whose distal ends 8 have a fixed first spacing 21.1 from one another and which are arranged immediately adjacent to the illumination fiber 7, but also two second detector fibers 14.2, which also have a fixed second spacing 21.2 from one another. In this case, the first spacing 21.1 is less than the second spacing 21.2. As already described above, the spacing 21 between the two detector fibers 14 used for the signal capture ultimately forms the stereo basis. In this case, the ratio of the stereo basis to the working distance 22 is a measure for the quality of the stereo impression, wherein a value of approximately 1:10 was found to be particularly advantageous in this context. As the working distance 22 is chosen to be larger, the stereo basis must also be chosen to be larger here. In the case of a small working distance 22 of for example 2 mm, as depicted in FIG. 5, it is possible to use the two first detector fibers 14.1, whose first spacing 21.1 is approximately 200 μm, for signal acquisition. By contrast, if the working distance 22 is increased to 10 mm, as indicated in FIG. 6, then it is possible to use the two second detector fibers 14.2 which have a second spacing 21.2 of approximately 1 mm from one another. In this case, suitable signal acquisition can ensure that the contribution of the first detector fibers 14.1 to the signal acquisition is suppressed in the case of a relatively large working distance 22, for example by virtue of possible scattered light 13 not being output coupled from the proximal ends 6 of the first detector fibers 14.1. For example, the working distance 22 can be detected here via OCT measurements in each case, the latter being performed through the illumination fiber 7. In the embodiment shown, it is also important to observe that the size of the raster-scannable object region 12 is variable, and the size of the object region 12 is adjusted automatically when the working distance 22 is changed in order to always obtain an unchanging image.

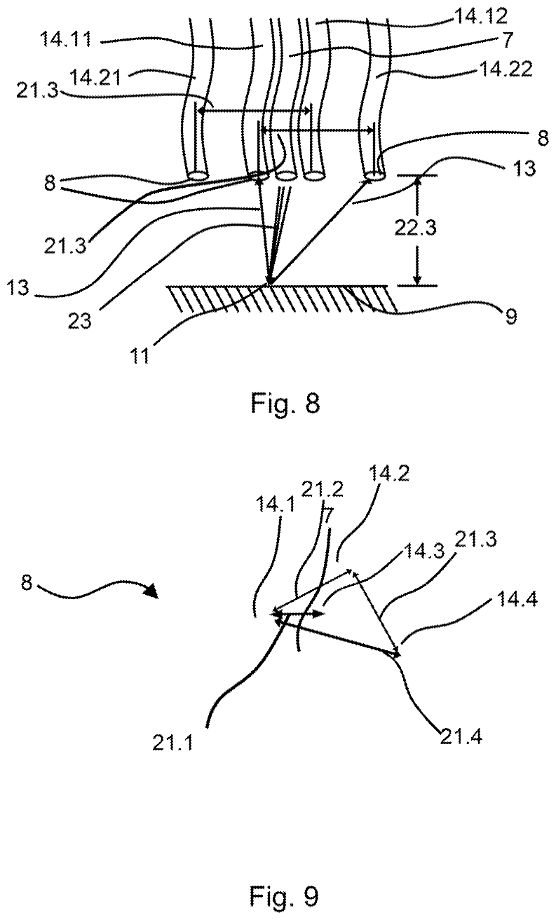

FIG. 8 shows a further detail view of the embodiment depicted in FIGS. 5 and 6. In this case, it is not the two first detector fibers 14.11 and 14.12 or the two second detector fibers 14.21 and 14.22 that are used for the signal acquisition; instead, a first detector fiber 14.11 and a second detector fiber 14.22 can be used together in each case. These have a fixed third spacing 21.3 which lies between the fixed first spacing 21.1 and the fixed second spacing 21.2.

As already described above, the spacing 21 between the two detector fibers 14 used for the signal capture ultimately forms the stereo basis. In this case, the ratio of the stereo basis to the working distance 22 is a measure for the quality of the stereo impression, wherein a value of approximately 1:10 was found to be particularly advantageous in this context. As the working distance 22 is chosen to be larger, the stereo basis must also be chosen to be larger here. If the fixed spacing 21.1 between the detector fibers 14.11 and 14.12 is approximately 200 μm and the fixed spacing 21.2 between the detector fibers 14.21 and 14.22 is approximately 1 mm, then the fixed spacing 21.3 between the detector fibers 14.11 and 14.22 or between the detector fibers 14.12 and 14.21 is approximately 600 μm, and so this combination of detector fibers can be used particularly well in the case of a working distance of approximately 6 mm. In a manner analogous thereto, the detection fibers 14.11 and 14.21 or 14.12 and 14.22 could also be used to realize a fourth fixed spacing. By preference, on account of the pre-shaping of the wavefront, the sample beam 23 emerging from the distal end 8 of the illumination fiber 7 is configured to raster-scan object points 11 which are located in a joint capturing region of the respectively used combination of fiber pairs. In an alternative or in addition, it would also be possible to use the capturing regions of the fiber pairs 14.11, 14.22 and 14.12 and 14.21 for signal acquisition and suitably combine, for example, join together or fuse, the respectively captured regions or acquired data.

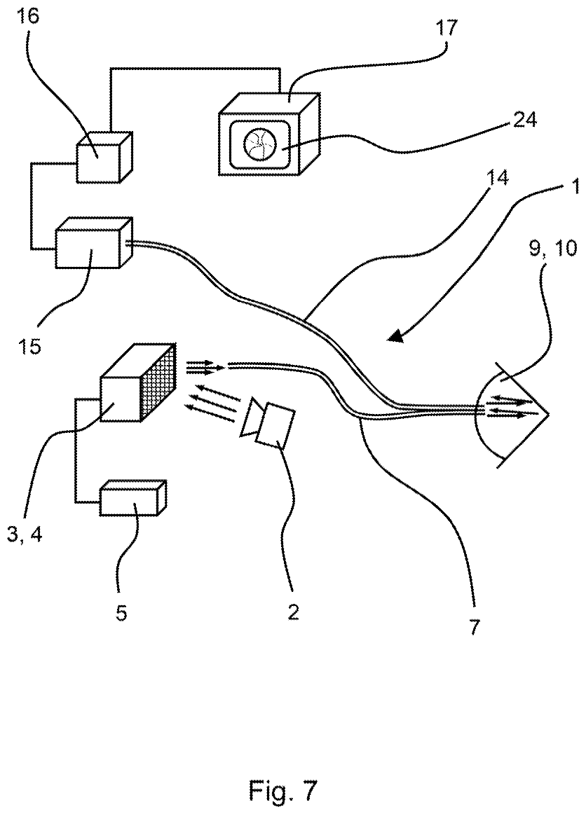

FIG. 7 shows a third embodiment of the fiberscope 1. As an alternative to the embodiments depicted in FIG. 1 and FIG. 4, it is possible in this case to also realize a stereoscopic fiberscope 1 which has only one illumination fiber 7 and only one detector fiber 14. In this case, the wavefront manipulator 3 is actuated such that all scanned object points 11 are always scanned by two sample beams 23 which make a defined stereo angle with each other, wherein this case also requires a temporal and/or spectral separation of the signal acquisition. However, in the case of spectral separation, it is then advantageous to use a second light source 2 with a second detector fiber 14, wherein the two light sources 2 emit light at different wavelengths. Suitable filter elements 18 prevent scattered light 13 from being guided to a non-corresponding detector 15.

FIG. 9 shows a detail view of the distal end 8 of a fourth embodiment of the fiberscope in cross section and can be considered to be a development of the aforementioned first to third embodiments, and hence can be combined in part or as a whole with these embodiments without restrictions. The distal ends of a plurality of fibers are depicted as concentric circles, with the fibers being arranged as a fiber bundle by way of example and the detector fibers 14.1-14.4 and an illumination fiber 7 being labelled by way of example. There is a fixed or unchanging spatial spacing between all fibers, especially the detector fibers, as depicted by way of example between some of the detector fibers 14.1-14.4, and this spacing is depicted by way of example as spacing 21.1-21.4 and can essentially depend on the dimensions of the individual fibers. In particular, it may be possible for detector fiber and illumination fiber to have a different diameter. In particular, a plurality of detector fibers and/or illumination fibers may also be arranged in a row.

In this case, the spacings between the individual distal ends of the detector fibers, shown by way of example as spacings 21.1-21.4 between the detector fibers 14.1-14.4, are different and ultimately form the stereo basis usable for the signal acquisition. The detector fibers 14.1-14.4 can be used in dynamic pairwise fashion for signal acquisition, especially depending on the working distance. Expressed differently, the stereo basis, and hence the pairs of detector fibers used for the signal acquisition, can be chosen on the basis of the working distance such that the ratio between the stereo basis and the working distance, which is a measure for the quality of the stereo impression, advantageously has a value of approximately 1:10. As the working distance is chosen to be larger, the stereo basis can also be chosen to be larger here. As a result of the arrangement of the detector fibers and the plurality of the detector fibers, the stereo basis can be set granularly, that is, with fine gradations, on the basis of the working distance, and so the preferred ratio between stereo basis and working distance of approximately 1:10 is kept substantially constant.

As an alternative or in addition, provision can also be made for a plurality of illumination fibers in the fiber bundle shown in FIG. 9 such that the illumination region can be increased in size and flexibly adjusted. The number of fibers shown in the fiber bundle shown in FIG. 9 should also only be understood to be by way of example; the number can be increased or reduced.

Furthermore, the signals recorded in particular also as a result of the multiplicity of possible combinations of fiber pairs can be used to make a 3-D reconstruction of the object region in order to provide the user of the fiberscope with a virtual 3-D image of the object region. This virtual image can inter alia also be augmented with other information, especially presurgical information or information from other imaging modalities.

In an alternative or in addition, provision can be made for detector fibers to also be used as illumination fibers and for illumination fibers to also be used as detector fibers. As a result of the use as illumination and/or detector fiber depending not on the configuration at the distal end but only on the configuration at the proximal end, the distal end of the endoscope can be kept small in terms of its dimensions and nevertheless be configured flexibly and dynamically and hence adjusted to the required or advantageous signal acquisition.

In an alternative or in addition, stereoscopic parameters of a user can also be used in the aforementioned embodiments to influence the ratio between stereo basis and working distance. For example, it may be advantageous to increase or reduce the size of the stereo basis as a result of specifications, inputs or requirements of a user. As a result, the stereo basis can be adjusted flexibly to the situational or user-specific requirements. In this context, stereoscopic parameters of a user may also include the eye spacing of the user. Furthermore, stereoscopic parameters of a user may also include values for the ratio of stereo basis to working distance used in the past, especially regularly recurring deviations from the ratio of 1:10, which was found to be sensible or advantageous, and averaged values or values calculated differently, and/or also situationally dependent values, advantageously also based on values used in the past, for example depending on the operation phase, the type of operation, the size of the object to be examined and/or the patient.

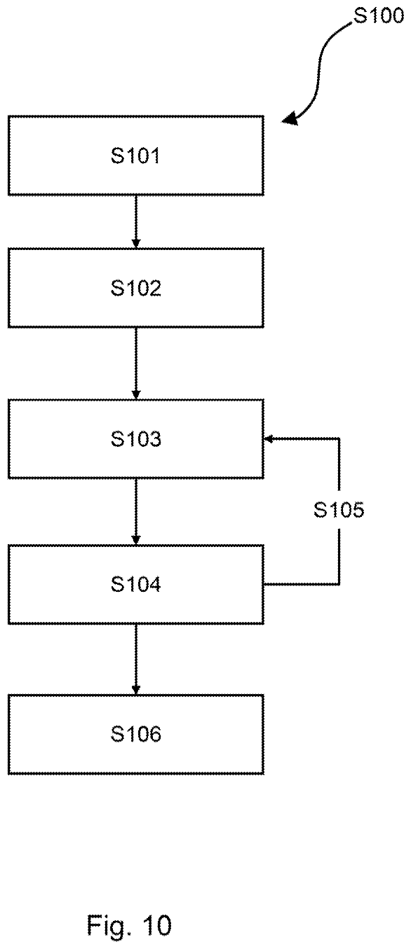

FIG. 10 shows a flowchart of a method S100 for acquiring stereoscopic image data from a fiberscope 1. In the method S100, a wavefront of the light from a light source 2 is initially pre-shaped via a wavefront manipulator 3 in a first step S101. In a further step S102, this pre-shaped wavefront is then supplied as a sample beam 23 to an object region 12, which ultimately represents the part of the examination object 9 illuminable by the fiber endoscope 1, via an illumination fiber 7. In so doing, the sample beam 23 is focused on an object point 11 in the object region 12 in a step S103. In a step S104, the scattered light 13 scattered and/or reflected by the respective object point 11 is supplied via a detector fiber 14 to a detector 15 which acquires the signal from the scattered light 13 and transmits this to a computer unit 16. Steps S103 and S104 are repeated S105 in this case for at least some of the object points 11 which form the object region. In a final step S106, the computer unit 16 ultimately acquires a stereoscopic image 24 from the scattered light 13 captured by the detector 15. As already explained above with reference to FIGS. 1 to 7, different combinations of illumination fibers 7 and detector fiber 14 can be used in the process. For example, if only one illumination fiber 7 and also only one detector fiber 14 are used, then it is necessary to always scan each object point 11 in the object region 12 by two sample beams 23 which make a defined stereo angle with each other. In this case, the capture of the scattered and/or reflected scattered light 13 is implemented in temporally and/or spectrally separated fashion by the detector 15 coupled to the detector fiber 14. Then, the stereoscopic image 24 can be extracted and composed, for example via the computer unit 16, from this temporally and/or spectrally separately captured scattered light 13. In a further embodiment, use is made of one illumination fiber 7 in combination with two detector fibers 14. In this case, the distal ends 8 of the two detector fibers 14 have a defined spacing 21 and ultimately form the stereo basis and thus input couple the scattered light 13 into the detector fibers 14 from different angles. In a further embodiment, each fiberscope 1 includes two illumination fibers 7 and two detector fibers 14. Here, too, the two detector fibers 14 have a fixed spacing 21 from one another, which ultimately forms the stereo basis. In this embodiment there is a temporal or spectral separation of the two detector fibers 14, as has already been described in detail above.

In a further embodiment, the fiberscope 1 includes one or more illumination fibers 7 and a plurality of detector fibers 14. Here, too, the detector fibers 14 have a fixed spacing 21 from one another, which ultimately forms the stereo basis. There may be dynamic pairing of detector fibers 14 and a temporal and/or spectral separation of detector fibers 14, as already described in detail above.

During the focusing in step S103, the working distance 22 between a distal end 8 of the illumination fiber 7 and the object region 12 is determined first; for example, this can be implemented via OCT measurements. The working distance 22 determined thus between the distal end 8 of the illumination fiber 7 and the object region 12 is then used to adjust the size of the object region 12.

In an alternative or in addition, the working distance determined thus can be used to select a combination of one or more detection fiber pairs and/or illumination fibers.

It is understood that the foregoing description is that of the preferred embodiments of the invention and that various changes and modifications may be made thereto without departing from the spirit and scope of the invention as defined in the appended claims.

### LIST OF REFERENCE SIGNS

1 Fiberscope
2 Light source
3 Wavefront manipulator
4 Micromirror actuator
5 Controller
6 Proximal end
7 Illumination fiber
8 Distal end
9 Examination object
10 Eye
11 Object point
12 Object region
13 Scattered light
14 Detector fiber
15 Detector
16 Computer unit
17 Display device
18 Filter element
19 Capturing region
20 Overlap region
21 Spacing
22 Working distance
23 Sample beam
24 Stereoscopic image
S100-S106 Method steps

1. A fiberscope for stereoscopic imaging, the fiberscope comprising:
at least one wavefront manipulator which, for creating a sample beam, is configured to pre-shape a wavefront of light from a light source such that the pre-shaped light is focusable on an object point in an object region and raster-deflectable to a multiplicity of object points;
an illumination fiber for supplying a pre-shaped sample beam to the object region;
a detector fiber for supplying scattered light at least one of reflected and scattered at a respective object point to a detector configured to capture the scattered light and being connected to a computer unit; wherein:
i) the wavefront manipulator further is configured to create at least one of temporally separated sample beams and spectrally separated sample beams, which make a fixed stereo angle with each other, or
ii) the wavefront manipulator, the illumination fiber and the detector fiber are each provided twice, with the wavefront manipulators further being configured to create at least one of the temporally separated sample beams and the spectrally separated sample beams; and,
the computer unit being configured to form the stereoscopic image from at least one of the captured temporally separated scattered light and the captured spectrally separated scattered light.

2. A fiberscope for stereoscopic imaging, the fiberscope comprising:
at least one wavefront manipulator which, for creating a sample beam, is configured to pre-shape a wavefront of light from a light source such that the pre-shaped light is focusable on an object point in an object region and raster-deflectable to a multiplicity of object points;
an illumination fiber for supplying a pre-shaped sample beam to the object region;
at least two detector fibers for supplying scattered light at least one of reflected and scattered at a corresponding object point to a corresponding detector configured to capture the scattered light and being connected to a computer unit; and,
the computer unit being configured to compose the stereoscopic image from the captured scattered light.

3. The fiberscope of claim 2, wherein the at least two detector fibers have a fixed lateral spacing from one another.

4. The fiberscope of claim 3, wherein the fixed lateral spacing between the at least two detector fibers lies between 50 μm and 500 μm.

5. The fiberscope of claim 3, wherein the fixed lateral spacing between the at least two detector fibers lies between 100 μm and 400 μm.

6. The fiberscope of claim 3, wherein the fixed lateral spacing between the at least two detector fibers lies between 150 μm and 300 μm.

7. The fiberscope of claim 3, wherein the fixed lateral spacing between the at least two detector fibers is 200 μm.

8. The fiberscope of claim 1, wherein the at least two detector fibers are arranged such that a same detection region is observable.

9. The fiberscope of claim 1, wherein a size of the object region able to be scanned is variable.

10. The fiberscope of claim 1, wherein the wavefront manipulator is configured to vary a size of the object region when a working distance is changed.

11. The fiberscope of claim 2, wherein the wavefront manipulator is configured to vary a size of the object region when a working distance is changed.

12. The fiberscope of claim 1, wherein the light source is provided twice.

13. The fiberscope of claim 2, wherein at least two of the illumination fiber and the at least two detector fibers are arranged immediately adjacent to one another.

14. The fiberscope of claim 1, wherein the illumination fiber and the detector fiber are arranged immediately adjacent to each other.

15. The fiberscope of claim 2, wherein at least two of the illumination fiber and the at least two detector fibers are arranged immediately adjacent to each other.

16. The fiberscope of claim 1 further comprising a display apparatus for displaying the stereoscopic image.

17. The fiberscope of claim 2 further comprising a display apparatus for displaying the stereoscopic image.

18. A method for acquiring stereoscopic image data from a fiberscope, the method comprising:
a) pre-shaping a wavefront of light from at least one light source via at least one wavefront manipulator such that, for creating a sample beam, pre-shaped light is focusable on an object point in an object region and can be raster-deflected to a multiplicity of object points;
b) supplying a pre-shaped sample beam to an object region via at least one illumination fiber;
c) focusing supplied light on the object point in the object region by way of the at least one wavefront manipulator;
d) supplying scattered light at least one of reflected and scattered at the object point to a detector via a detector fiber;
e) repeating steps c) and d) for at least a subset of the object points in the object region; and,
f) extracting a stereoscopic image from the acquired data of the detector.

19. The method of claim 18, wherein said focusing in step c) is preceded by a determination of a working distance between a distal end of the at least one illumination fiber and the object region.

20. The method of claim 19, wherein the working distance between the distal end of the at least one illumination fiber and the object region is used to adjust a size of the object region.
